(19) United States
(12) Patent Application Publication
(10) Pub. No.: US 2023/0041556 A1
(43) Pub. Date: Feb. 9, 2023

(54) COLONIC PURGATIVE COMPOSITION

(71) Applicant: Vivozon, Inc., Gyeonggi-do (KR)

(72) Inventor: Il Kyu YOON, Gyeonggi-do (KR)

(21) Appl. No.: 17/787,998

(22) PCT Filed: Dec. 22, 2020
(86) PCT No.: PCT/KR2020/018844

§ 371 Date: Jun. 22, 2022

(30) Foreign Application Priority Data
Dec. 23, 2019 (KR) 10-2019-0172915

(51) Int. Cl. A61K 33/04; A61K 33/06; A61K 31/80; A61P 1/10; A61K 9/16; A61K 9/20; A61K 9/28

(52) U.S. Cl. CPC A61K 33/04; A61K 33/06; A61K 31/80; A61P 1/10; A61K 9/1635; A61K 9/2027; A61K 9/2031; A61K 9/2833

(57) ABSTRACT

The present invention relates to a colonic purgative composition and a preparation method therefor, and more specifically, to a colonic purgative composition comprising sodium picosulfate, potassium sulfate and magnesium sulfate, and a preparation method therefor.

### TECHNICAL FIELD

The present invention relates to a colonic purgative composition and a method for preparing the same, and more particularly, to a colonic purgative composition comprising sodium picosulfate, potassium sulfate and magnesium sulfate, and a method for preparing the same.

### BACKGROUND ART

Colonoscopy and bowel surgery require pretreatment to completely remove intestinal remnants prior to implementation. In order to effectively perform pretreatment, various types of colonic purgatives have been developed. Colonic purgatives can be classified into osmotic purgatives, stimulatory purgatives, emollient purgatives and the like.

Osmotic purgatives are non-absorbable electrolyte solutions that create a difference in osmotic pressure in the intestine after ingestion to wash out feces. Osmotic purgatives are further classified into hyperosmotic purgatives and saline purgatives.

As a hyperosmotic purgative, a polyethylene glycol (PEG)-electrolyte solution is used. However, due to its high dosage (4 liter solution) and the characteristic odor and salty taste, compliance with the medication is poor in most patients. As a result, patients cannot take all of the pretreatment solution, which results in poor colon cleanliness during the examination.

Examples of saline purgatives are phosphates and sulfates. Phosphate has a relatively low dose (2 liters of water) and high patient compliance, but may lead to electrolyte abnormalities and is contraindicated in patients with heart failure or advanced liver or kidney disease. In 2008, the U.S. Food and Drug Administration (FDA) reported the occurrence of “acute phosphate nephropathy,” one of the acute kidney injuries when using “oral sodium phosphate formulations.” Accordingly, in 2013, Korea's Ministry of Food and Drug Safety also restricted the use of phosphate formulations as purgatives for colonoscopy. Sulfate is evaluated to be relatively safer than phosphate, and as the formulations, sodium sulfate, potassium sulfate, magnesium sulfate and the like are mainly used. The dosage has been greatly reduced, but it still needs to be reduced, and in particular, due to the salty taste of sodium sulfate, it is necessary to improve adherence to medication.

Stimulant purgatives interfere with the absorption of water and electrolytes in the large intestine and stimulate the intestinal mucosa to induce contraction of the colon muscles to force defecation. Examples of stimulant purgatives include a combination of sodium picosulfate, magnesium oxide and citric acid.

In the case of the existing liquid form of large intestine purgatives (liquid formulation), 4 liters or 2 liters (additional water intake) must be taken, so women or the elderly have a burden of drinking a lot of water. In the case of powders, the preparation method of a purgative by diluting it with water is complicated, and due to the unpleasant taste of the main ingredient, even if it is possible to take it to some extent in the beginning, at the end of administration, it may cause nausea or vomiting. Due to such nausea or vomiting, purgatives cannot be taken properly, which may cause a decrease in the screening rate of colonoscopy.

On the other hand, there have been efforts to develop a solid formulation in order to solve problems such as the possibility of deterioration of the liquid formulation, the use of an excessive amount of preservatives and sweeteners, and an increase in distribution costs. For example, Korean Laid-Open Patent Application No. 10-2015-0089430 discloses an intestinal purgative composition comprising magnesium sulfate, potassium sulfate and sodium sulfate as active ingredients, polyethylene glycol as an excipient and sodium stearyl fumarate as a lubricant. Although there is no clear reference to the dosage of the active ingredient in the above invention, it discloses that in order to maximize the convenience of taking the purgative and its effect, each tablet is divided into two divided doses and taken within 1 hour at 15-minute intervals when taken once and by adjusting the tablet size—for example, if you take the purgative only the day before colonoscopy, you can take 3 tablets at a time for a total of 15 tablets, 4 tablets at a time for a total of 20 tablets, 5 tablets at a time for a total of 25 tablets and 6 tablets at a time for a total of 30 tablets. It is understood that there is practically no difference in the dosage of the liquid formulation and the active ingredient. It is difficult to expect improvement in medication compliance because a large amount of water is required every time many tablets are taken.

In addition, Korean Laid-Open Patent Publication No. 10-2019-0041233 discloses a composition for a colonic purgative comprising magnesium sulfate anhydrous, potassium sulfate, sodium sulfate anhydrous and simethicone. The above invention discloses that when implemented as a tablet, one tablet may comprise 102.86 mg of magnesium sulfate anhydrous, 201.07 mg of potassium sulfate, 1,125.00 mg of sodium sulfate anhydrous and 11.43 mg of simethicone, and 14 tablets and 1,277 ml of water are taken within 1 hour the day before the test, and 14 tablets and 1,277 ml of water can be taken within 1 hour on the morning of the test day, so a total of 28 tablets need to be taken before the test.

### CONTENTS OF THE INVENTION

### Problems to be Solved

The purpose of the present invention is to provide a colonic purgative composition having an improved colon cleansing effect and improved medication compliance.

In addition, the purpose of the present invention is to provide a method for preparing a colonic purgative composition with easy quality control.

### TECHNICAL MEANS

In order to achieve the technical purpose, the present invention provides a colonic purgative composition comprising sodium picosulfate, potassium sulfate and magnesium sulfate.

In addition, the present invention provides a solid formulation for oral administration comprising the above colonic purgative composition.

In addition, the present invention provides a method for preparing a colonic purgative composition, comprising mixing a first mixture comprising potassium sulfate, magnesium sulfate and simethicone; and a second mixture comprising sodium picosulfate, a water-soluble binder and a solvent to prepare a granulated product.

Here in after, the present invention will be explained in detail.

According to one aspect of the present invention, there is provided a colonic purgative composition comprising sodium picosulfate, potassium sulfate and magnesium sulfate.

In the colonic purgative composition according to the present invention, by combining potassium sulfate and magnesium sulfate, which are saline purgatives, with sodium picosulfate, which is a stimulant purgative, an excellent colon cleansing effect can be exhibited while reducing the dosage compared to conventional sulfate formulations.

In one embodiment according to the present invention, the colonic purgative composition may further comprise simethicone. Simethicone is an anti-foaming agent used to prevent bubbles from forming in an endoscopy, and when the colonic purgative composition according to the present invention additionally comprises simethicone, the visual field of the endoscope can be improved.

In another embodiment according to the present invention, the magnesium sulfate comprised in the colonic purgative composition may be magnesium sulfate tetrahydrate. Magnesium sulfate exists in various forms, from the anhydrate form to the heptahydrate form. When magnesium sulfate tetrahydrate is used in the present invention, it may be more advantageous in terms of minimizing tabletting disorders (e.g., sticking, capping, etc.) during tablet production compared to heptahydrate. In addition, when magnesium sulfate tetrahydrate is used in the present invention, it may be more advantageous because the preparation process, wetting of the final drug and the resulting exothermic reaction are minimized compared to anhydrous. If the intermediate product or the final product is wetted during the preparation process, it may cause difficulties in standard control due to swelling. In particular, if the final product is moistened in body organs after taking it, organs may be damaged due to heat.

In another embodiment according to the present invention, the content of each component of the colonic purgative composition in the total dose before colonoscopy may be:

Sodium picosulfate: a daily dose of 7.5 to 10.5 mg (×2 days)

Potassium sulfate: a daily dose of 2.5 to 3.3 g (×2 days)

Magnesium sulfate tetrahydrate: a daily dose of 5.7 to 9 g (×2 days)

Simethicone: a daily dose of 140 to 200 mg (×2 days).

In another embodiment according to the present invention, the content of each component of the colonic purgative composition in the total dose before colonoscopy may be:

Sodium picosulfate: a daily dose of 7.5 to 10 mg (×2 days)

Potassium sulfate: a daily dose of 2.5 to 3.13 g (×2 days)

Magnesium sulfate tetrahydrate: a daily dose of 5.72 to 8.58 g (×2 days)

Simethicone: a daily dose of 160 to 200 mg (×2 days).

In another embodiment according to the present invention, the colonic purgative composition may further comprise a water-soluble binder, a water-soluble lubricant, or both a water-soluble binder and a water-soluble lubricant.

In another embodiment according to the present invention, the water-soluble binder may be selected from the group consisting of polyvinylpyrrolidone, copolymers of vinylpyrrolidone and vinyl acetate, polyethylene glycol and combinations thereof. As polyvinylpyrrolidone, for example, povidone or kollidon may be used. As the copolymer of vinylpyrrolidone and vinyl acetate, for example, copovidone such as Kollidon VA64 may be used.

In another embodiment according to the present invention, the water-soluble lubricant may be selected from the group consisting of sodium benzoate, sodium lauryl sulfate and combinations thereof. In another embodiment according to the present invention, the water-soluble lubricant may be sodium benzoate.

In another embodiment according to the present invention, the colonic purgative composition may further comprise a water-soluble antioxidant in addition to a water-soluble binder and/or a water-soluble lubricant.

In another embodiment according to the present invention, the water-soluble antioxidant may be selected from the group consisting of ascorbic acid, sodium ascorbate, fumaric acid, malic acid, potassium metabisulfite, sodium pyrosulfite and combinations thereof.

In another embodiment according to the present invention, the colonic purgative composition may be formulated in the form of a solution for binding or mixing the components with water, a solid formulation such as tablets or capsules, especially solid formulation for oral administration or a semi-solid (e.g., a gel).

According to one aspect of the present invention, there is provided a solid formulation for oral administration comprising the colonic purgative composition.

In another embodiment according to the present invention, the solid formulation for oral administration may further comprise a coating layer. In the present invention, when the solid formulation for oral administration additionally comprises a coating layer, it is possible to mask the characteristic taste of sulfate. In another embodiment according to the present invention, the coating layer may be selected from the group consisting of polyvinyl alcohol-polyethylene glycol graft copolymer, amino methacrylate copolymer, polyvinyl alcohol, copolymer of polyethylene glycol and methacrylate, and combinations thereof.

In another embodiment according to the present invention, the total dose of sodium picosulfate, potassium sulfate and magnesium sulfate before endoscopy may be 22.26 to 24.62 g in the solid formulation for oral administration. In another embodiment according to the present invention, the total dose of sodium picosulfate, potassium sulfate, magnesium sulfate and simethicone before endoscopy is 22.57 to 24.95 g in the solid formulation for oral administration.

In another embodiment according to the present invention, in the solid preparation for oral administration, the total dose of sodium picosulfate before endoscopy may be 19 to 21 mg. In another embodiment according to the present invention, in the solid preparation for oral administration, the total dose of potassium sulfate before endoscopy may be 5,947 to 6,573 mg. In another embodiment according to the present invention, in the solid preparation for oral administration, the total dose of magnesium sulfate before endoscopy may be 16.3 to 18.02 g. In another embodiment according to the present invention, in the solid formulation for oral administration, the total dose of simethicone before endoscopy may be 304 to 336 mg.

In another embodiment according to the present invention, the solid formulation for oral administration may be administered in divided doses once on the day before the endoscopy and once on the day of the examination. In another embodiment according to the present invention, the solid formulation for oral administration may be administered in divided doses of 10 tablets the day before the endoscopy and 10 tablets on the day of the endoscopy. In another embodiment according to the present invention, the solid formulation for oral administration may be administered in doses of 10 tablets (3 to 5 divided doses) with water (425 mL) and 2 additional doses of water (425 mL) within 60 minutes may be administered on the day before the endoscopy, and on the morning of the test, the solid formulation for oral administration may be administered in doses of 10 tablets (3 to 5 divided doses) with water (425 mL) and then 2 additional doses of water (425 mL) within 60 minutes may be administered.

In another embodiment according to the present invention, the solid formulation for oral administration may comprise 1 mg of sodium picosulfate, 313 mg of potassium sulfate, 858 mg of magnesium sulfate tetrahydrate and 16 mg of simethicone per tablet. Referring to FIG. 3, the tablet may be manufactured in a smaller size (16 mm long axis, 9 mm short axis) than conventional laxative tablets. Therefore, as the dose is reduced, the convenience of taking the dose increases in terms of the number of doses and the size.

According to another aspect of the present invention, there is provided a method for preparing a colonic purgative composition, comprising mixing a first mixture comprising potassium sulfate, magnesium sulfate and simethicone; and a second mixture comprising sodium picosulfate, a water-soluble binder and a solvent.

In another embodiment according to the present invention, the water-soluble binder may be selected from the group consisting of polyvinylpyrrolidone, copolymers of vinylpyrrolidone and vinyl acetate, polyethylene glycol and combinations thereof.

In another embodiment according to the present invention, the solvent may be ethanol.

### Effect of the Invention

The colonic purgative composition according to the present invention not only does not have the characteristic odor or taste of the colonic purgative, but also can provide an excellent colon cleansing effect while significantly improving medication compliance due to a reduced dosage.

### BRIEF DESCRIPTION OF THE FIGURES

FIG. 1 is a result confirming the colon cleansing effect of the colonic purgative composition of Example 1 in an animal model.

FIG. 2 is a result showing the state of the colon when the colonic purgative composition is not administered in an animal model.

FIG. 3 is a cross-sectional view of the coating agent of Example 12.

FIG. 4 is a photograph taken by excising the colon portion of the rat administered the composition of Example 9.

FIG. 5 is a photograph taken by excising the colon portion of the rat administered with the composition of the Comparative Example.

### CONCRETE MODE FOR CARRYING OUT THE INVENTION

Hereinafter, the present invention will be explained in detail with reference to the following Examples. However, these Examples are only meant to illustrate the invention and its scope, and are not limited thereto in any manner.

### Preparation Example: Preparation of Magnesium Sulfate Tetrahvdrate

100 g of magnesium sulfate heptahydrate was evenly spread on the plate and dried in a dryer at 50±5° C. After a certain period of time, the dried product was obtained and used when 22 g of moisture was dried compared to the initial weight—that is, when the net weight reached 78 g.

### Example 1: Preparation of Colonic Purgative Composition

10 mg of sodium picosulfate, 3.13 g of potassium sulfate and 8.58 g of magnesium sulfate tetrahydrate were dissolved in water to prepare a solution of 20 ml.

### Experimental Example 1: Colon Cleansing Effect of the Colonic Purgative Composition

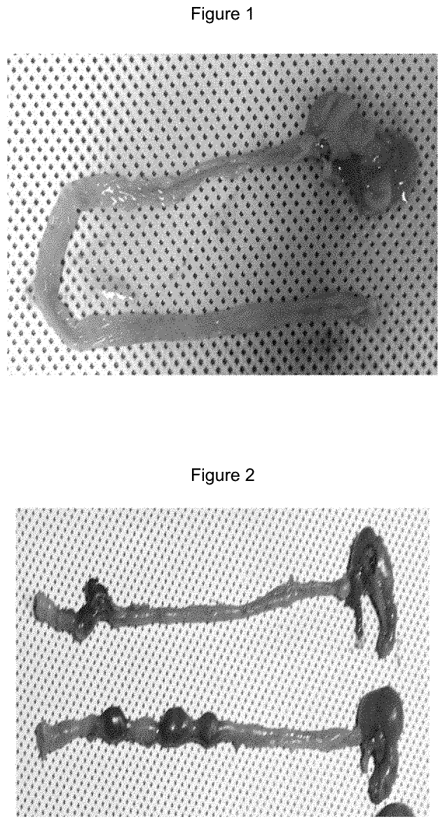

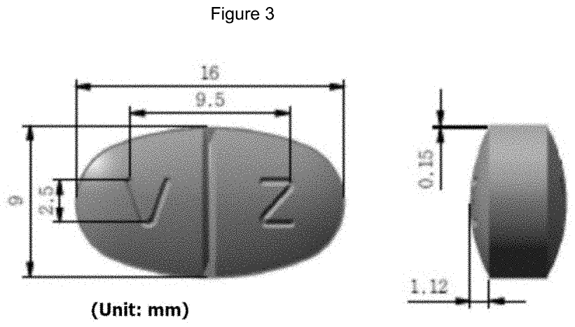

The colon cleansing effect of the composition of Example 1 was confirmed using an animal model. The composition of Example 1 was administered to rats. After 6 hours of initiation of administration, they were exsanguinated and killed. Thereafter, as a result of excising the colon and confirming the degree of colon cleansing, it was confirmed that the feces present in the colon were well removed in the group administered with the composition of Example 1 (FIG. 1). On the other hand, in the case of the group not administered the colon cleansing solution, it was confirmed that feces were present in the colon as it is (FIG. 2).

### Examples 2 to 4: Preparation of Sized Product of Colonic Purgative

First, potassium sulfate, magnesium sulfate (tetrahydrate) and simethicone were weighed as shown in Table 1 below, put into a mixer and mixed evenly to prepare a first mixture. A second mixture was prepared by dissolving sodium picosulfate and povidone as a binder in 1.0 g of purified water or ethanol. The second mixture was put into the mixer and granulated for 3 minutes. The obtained granulated material was dried at 55° C. for 2 hours. After the dried granulated material was sieved through a 0.8 mm sieve of, the weight of the obtained sized product was checked. Table 1 shows the weight increase rate of the obtained sized product compared to the total weight of the components added except for purified water or ethanol.

TABLE 1

| | Example 2 | Example 3 | Example 4 |
|---|---|---|---|
| First mixture | Potassium sulfate 3.13 g | Potassium sulfate 3.13 g | Potassium sulfate 3.13 g |
| | Magnesium sulfate 8.58 g | Magnesium sulfate 8.58 g | Magnesium sulfate 8.58 g |
| | Simethicone 0.16 g | Simethicone 0.16 g | Simethicone 0.16 g |
| Second mixture | Sodium picosulfate 0.01 g | Sodium picosulfate 0.01 g | Sodium picosulfate 0.01 g |
| | Binder (povidone) 0.39 g | Binder 0.39 g | Binder 0.39 g |
| | Purified water 1.0 g | 50% ethanol 1.0 g | 95% ethanol 1.0 g |
| Weight of the components | 12.27 g | 12.27 g | 12.27 g |
| added (except water, | | | |
| purified water) | | | |
| Weight of sized product | 12.76 g-12.86 g | 12.46 g-12.56 g | 12.27 g-12.33 g |
| Weight increase rate | 4.0%-4.8% | 1.5%-2.4% | 0%-0.5% |

The sized products of Examples 2 and 3 were impregnated with magnesium by purified water and their weights increased by up to 4.8%. However, in the sized product of Example 4 obtained using ethanol, the water content was controlled to within 0.5%. Therefore, it was confirmed that the preparing process of Example 4 can be usefully used for mass production industrially.

### Example 5: Preparation of Uncoated Tablets for Colonic Purgative 1

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of povidone in 1.0 g of 95% ethanol. The binder solution was added to the mixer, granulated for 3 minutes and then dried at 55° C. for 2 hours. The dried granules were sieved through a 0.8 mm sieve to obtain a sized product. The sized product was tableted with a hardness of 10 to 15 Kp and a weight of 1,227 mg per tablet with an oval punch, with a major axis of 16.0 mm and a minor axis of 9.0 mm in a rotary tablet machine.

### Example 6: Preparation of Uncoated Tablets for Colonic Purgative 2

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.25 g of copovidone in 1.0 g of 95% ethanol. The binder solution was added to the mixer, granulated for 3 minutes and then dried at 55° C. for 2 hours. The dried granules were sieved through a 0.8 mm sieve to obtain a sized product. The sized product was tableted with a hardness of 10 to 15 Kp and a weight of 1,213 mg per tablet with an oval punch, with a major axis of 16.0 mm and a minor axis of 9.0 mm in a rotary tablet machine.

### Example 7: Preparation of Uncoated Tablets for Colonic Purgative 3

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of polyethylene glycol in 1.0 g of 95% ethanol. The binder solution was added to the mixer, granulated for 3 minutes and then dried at 55° C. for 2 hours. The dried granules were sieved through a 0.8 mm sieve to obtain a sized product. The sized product was tableted with a hardness of 10 to 15 Kp and a weight of 1,227 mg per tablet with an oval punch, with a major axis of 16.0 mm and a minor axis of 9.0 mm in a rotary tablet machine.

### Example 8: Preparation of Uncoated Tablets for Colonic Purgative 4

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of povidone in 1.0 g of 95% ethanol. The binder solution was added to the mixer, granulated for 3 minutes and then dried at 55° C. for 2 hours. The dried granules were sieved through a 0.8 mm sieve to obtain a sized product. After adding 0.25 g of copovidone to the sized product and mixing, the mixture was tableted with a hardness of 10 to 15 Kp and a weight of 1,252 mg per tablet with an oval punch, with a major axis of 16.0 mm and a minor axis of 9.0 mm in a rotary tablet machine.

### Example 9: Preparation of Uncoated Tablets for Colonic Purgative 5

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of povidone in 1.0 g of 95% ethanol. The binder solution was added to the mixer, granulated for 3 minutes and then dried at 55° C. for 2 hours. The dried granules were sieved through a 0.8 mm sieve to obtain a sized product. After mixing 0.25 g of copovidone and 0.32 g of sodium benzoate with the sized product in turn, the mixture was tableted with a hardness of 10 to 15 Kp and a weight of 1,284 mg per tablet with an oval punch, with a major axis of 16.0 mm and a minor axis of 9.0 mm in a rotary tablet machine.

### Example 10: Preparation of Uncoated Tablets for Colonic Purgative 6

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of povidone in 1.0 g of 95% ethanol. The binder solution was added to the mixer, granulated for 3 minutes and then dried at 55° C. for 2 hours. The dried granules were sieved through a 0.8 mm sieve to obtain a sized product. After mixing 0.25 g of copovidone and 0.03 g of sodium lauryl sulfate with the sized product in turn, the mixture was tableted with a hardness of 10 to 15 Kp and a weight of 1,255 mg per tablet with an oval punch, with a major axis of 16.0 mm and a minor axis of 9.0 mm in a rotary tablet machine.

### Example 11: Preparation of Uncoated Tablets for Colonic Purgative 7

3.13 g of potassium sulfate, 8.58 g of magnesium sulfate tetrahydrate and 0.16 g of simethicone were placed in a mixer and mixed evenly. A binding solution was prepared by dissolving 0.01 g of sodium picosulfate and 0.39 g of povidone in 1.0 g of 95% ethanol. The binder solution was added to the mixer, granulated for 3 minutes and then dried at 55° C. for 2 hours. The dried granules were sieved through a 0.8 mm sieve to obtain a sized product. After mixing 0.25 g of copovidone and 0.32 g of polyethylene glycol with the sized product in turn, the mixture was tableted with a hardness of 10 to 15 Kp and a weight of 1,284 mg per tablet with an oval punch, with a major axis of 16.0 mm and a minor axis of 9.0 mm in a rotary tablet machine.

TABLE 2

| | Example | | Example | | Example | | Example | | Example | | Example | | Example | |
|---|---|---|---|---|---|---|---|---|---|---|---|---|---|---|
| | 5 | | 6 | | 7 | | 8 | | 9 | | 10 | | 11 | |
| Sodium picosulfate | 10 | mg | 10 | mg | 10 | mg | 10 | mg | 10 | mg | 10 | mg | 10 | mg |
| Potassium sulfate | 3.13 | g | 3.13 | g | 3.13 | g | 3.13 | g | 3.13 | g | 3.13 | g | 3.13 | g |
| Magnesium sulfate * | 8.58 | g | 8.58 | g | 8.58 | g | 8.58 | g | 8.58 | g | 8.58 | g | 8.58 | g |
| Simethicone | 0.16 | g | 0.16 | g | 0.16 | g | 0.16 | g | 0.16 | g | 0.16 | g | 0.16 | g |
| Sodium pyrosulfite | | | | | | | | | 10 | mg | | | | |
| Povidone | 0.39 | g | — | | — | | 0.39 | g | 0.38 | g | 0.39 | g | 0.39 | g |
| Copovidone | — | | 0.25 | g | — | | 0.25 | g | 0.25 | g | 0.25 | g | 0.25 | g |
| Polyethylene glycol | — | | — | | 0.39 | g | — | | — | | — | | 0.32 | |
| Sodium benzoate | — | | — | | — | | — | | 0.32 | g | — | | — | |
| Sodium lauryl | — | | — | | — | | — | | — | | 0.03 | g | — | |
| sulfate | | | | | | | | | | | | | | |
| 95% ethanol | 1.0 | g | 1.0 | g | 1.0 | g | 1.0 | g | 1.0 | g | 1.0 | g | 1.0 | g |
| Weight of uncoated | 12.27 | g | 12.13 | g | 12.27 | g | 12.52 | g | 12.84 | g | 12.55 | g | 12.84 | g |
| tablet | | | | | | | | | | | | | | |

### Experimental Example 2: Evaluation of Uncoated Tablet

Tableting was evaluated for the mixture before tableting, tableting disturbances (sticking, capping, etc.) were checked during production of uncoated tablets, and the friability was measured according to the friability test method of tablets of the Korean Pharmacopoeia. As a result of the evaluation, all Examples showed good tabletting and friability. In particular, the uncoated tablet of Example 9 using sodium benzoate as a water-soluble lubricant showed the best tabletting performance and friability of 0.1% or less.

### Example 12: Preparation of Coated Tablets for Colonic Purgative

Polyvinyl alcohol-polyethylene glycol graft copolymer was dissolved in purified water at a concentration of 10% (w/w) to prepare a coating solution. 500 uncoated tablets of Example 9 were taken and placed in a coating machine, and then the coating solution was spray-dried under the following conditions to prepare coated tablets.

Coating equipment: Sejong C30JC

Inlet air temperature: 50° C.

Outlet air temperature: 40° C.

Fan rotation speed: 10 to 20 rpm

Spraying rate: 0.5 ml/min

### Experimental Example 3: Sensory Evaluation of Uncoated Tablets and Coated Tablets

For 10 people, taste evaluation was performed when taking the uncoated tablet of Example 9 and the coated tablet of Example 12, and the results are shown in Table 3.

TABLE 3

| Categories | Example 9 | Example 12 |
|---|---|---|
| Very salty | — | — |
| A little salty | — | — |
| Normal | 4 | — |
| Tasteless | 6 | 10 |
| Sum | 10 | 10 |

As can be seen from Table 3, even in the case of the uncoated tablet of Example 9, sodium sulfate, which accounts for the largest proportion of the existing sulfate complexes (magnesium sulfate, potassium sulfate, sodium sulfate), was replaced with sodium picosulfate, so that the salty taste and unpleasant odor of sodium sulfate could be reduced. In the case of the coated tablet of Example 12, since almost no salty taste was discerned, a significant improvement in medication compliance could be expected.

### Experimental Example 4: Comparison of Colon Cleansing Effect of Colon Purgative Composition in Animal Model

In order to compare the difference in colon cleansing effect, the colon cleansing effects of the composition of the above Example 9 and the composition of Example 3 disclosed in Korean Laid-Open Patent Application No. 10-2019-0142620 as a comparative example were confirmed using an animal model.

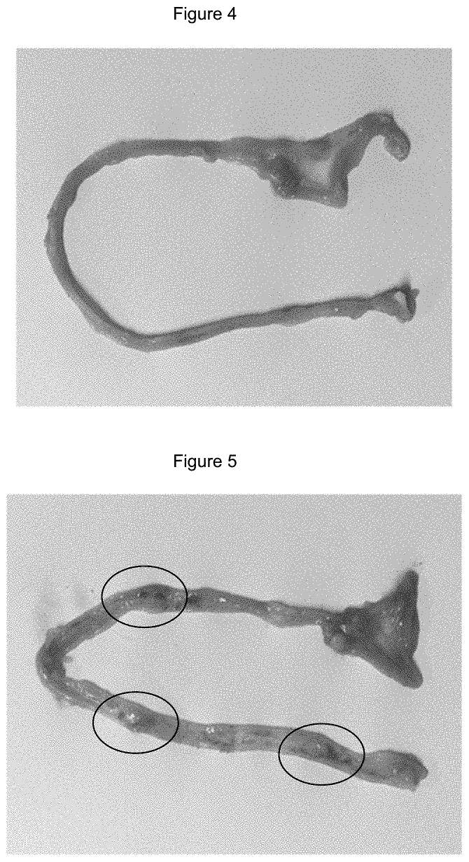

Specifically, the dose corresponding to the day before the test and on the day of the test was dissolved in 1,000 mL, of which 20 mL of the colon purgative composition of Table 4 was administered to the rats. After 6 hours of initiation of administration, they were exsanguinated and killed. Then, as a result of excising the colon and confirming the degree of colon washing, it was confirmed that the feces present in the colon were well removed in the group administered with the composition of Example 9 (FIG. 4). On the other hand, it was confirmed that the composition of the Comparative Example had residual stool in a portion of the colon (FIG. 5).

TABLE 4

| | Example 9 | | Comparative Example | |
|---|---|---|---|---|
| Sodium picosulfate | 0.4 | mg | — | |
| Sodium sulfate anhydrous | — | | 630.0 | mg |
| Potassium sulfate | 125.2 | mg | 112.6 | mg |
| Magnesium sulfate tetra- | 343.2 | mg | — | |
| hydrate | | | | |
| Magnesium sulfate anhydrate | — | | 57.6 | mg |
| Simethicone | 6.4 | mg | 6.4 | mg |
| Sodium pyrosulfite | 0.4 | mg | — | |
| Povidone | 15.2 | mg | — | |
| Copovidone | 10.0 | mg | Appropriate amount | |
| Sodium benzoate | 12.8 | mg | — | |
| Kollicoat IR | 10.4 | mg | Appropriate amount | |
| Purified water | 20 | mL | 20 | mL |

| | | |
|---|---|---|
| Total | 524.0 mg/20 mL | 840.0 mg/20 mL |

1. A colonic purgative composition comprising sodium picosulfate, potassium sulfate and magnesium sulfate.

2. The colonic purgative composition according to claim 1, further comprising simethicone.

3. The colonic purgative composition according to claim 2, wherein the magnesium sulfate is magnesium sulfate tetrahydrate.

4. The colonic purgative composition according to claim 3, wherein the content of each component in the total dose before colonoscopy is:
Sodium picosulfate: a daily dose of 7.5 to 10.5 mg
Potassium sulfate: a daily dose of 2.5 to 3.3 g
Magnesium sulfate tetrahydrate: a daily dose of 5.7 to 9 g
Simethicone: a daily dose of 140 to 200 mg.

5. The colonic purgative composition according to claim 4, wherein the content of each component in the total dose before colonoscopy is:
Sodium picosulfate: a daily dose of 7.5 to 10 mg
Potassium sulfate: a daily dose of 2.5 to 3.13 g
Magnesium sulfate tetrahydrate: a daily dose of 5.72 to 8.58 g
Simethicone: a daily dose of 160 to 200 mg.

6. The colonic purgative composition according to claim 3, further comprising a water-soluble binder and/or a water-soluble lubricant.

7. The colonic purgative composition according to claim 6, wherein the water-soluble binder is selected from the group consisting of polyvinylpyrrolidone, copolymers of vinylpyrrolidone and vinyl acetate, polyethylene glycol and combinations thereof.

8. The colonic purgative composition according to claim 6, wherein the water-soluble lubricant is selected from the group consisting of sodium benzoate, sodium lauryl sulfate and combinations thereof.

9. (canceled)

10. The colonic purgative composition according to claim 6, further comprising a water-soluble antioxidant.

11. The colonic purgative composition according to claim 10, wherein the water-soluble antioxidant is selected from the group consisting of ascorbic acid, sodium ascorbate, fumaric acid, malic acid, potassium metabisulfite sodium pyrosulfite, and combinations thereof.

12. A solid formulation for oral administration comprising the colonic purgative composition according to claim 1.

13. The solid formulation for oral administration according to claim 12, further comprising a coating layer.

14. The solid formulation for oral administration according to claim 13, wherein the coating layer is selected from the group consisting of polyvinyl alcohol-polyethylene glycol graft copolymer, amino methacrylate copolymer, polyvinyl alcohol, copolymer of polyethylene glycol and methacrylate, and combinations thereof.

15. The solid formulation for oral administration according to claim 12, wherein the total dose of sodium picosulfate, potassium sulfate and magnesium sulfate before endoscopy is 22.26 to 24.62 g.

16. The solid formulation for oral administration according to claim 12, wherein the total dose of sodium picosulfate, potassium sulfate, magnesium sulfate and simethicone before endoscopy is 22.57 to 24.95 g.

17. The solid formulation for oral administration according to claim 12, which is administered in divided doses once on the day before the endoscopy and once on the day of the endoscopy.

18. The solid formulation for oral administration according to claim 12, which is administered in divided doses of 10 tablets the day before the endoscopy and 10 tablets on the day of the endoscopy.

19. A method for preparing a colonic purgative composition, comprising mixing a first mixture comprising potassium sulfate, magnesium sulfate and simethicone; and a second mixture comprising sodium picosulfate, a water-soluble binder and a solvent.

20. The method for preparing a colonic purgative composition according to claim 19, wherein the water-soluble binder is selected from the group consisting of polyvinylpyrrolidone, copolymers of vinylpyrrolidone and vinyl acetate, polyethylene glycol and combinations thereof.

21. The method for preparing a colonic purgative composition according to claim 19, wherein the solvent is ethanol.
